(19) United States
(12) Patent Application Publication
(10) Pub. No.: US 2022/0288212 A1
(43) Pub. Date: Sep. 15, 2022

(54) FULLERENE PHOSPHONATE GALLOYLS AND METHODS

(71) Applicant: Sinapu LLC, Monrovia, CA (US)

(72) Inventor: Peter Butzloff, Walnut, CA (US)

(21) Appl. No.: 17/592,899

(22) Filed: Feb. 4, 2022

Related U.S. Application Data
(63) Continuation-in-part of application No. PCT/US22/14454, filed on Jan. 28, 2022.
Application No. PCT/US22/14454 is a continuation-in-part of application No. PCT/US2022/012369, filed on Jan. 13, 2022.
(60) Provisional application No. 63/161,310, filed on Mar. 15, 2021.

(51) Int. Cl. A61K 47/54; A61K 31/353; A61K 9/51; A61K 47/55

(52) U.S. Cl. CPC A61K 47/54; A61K 31/353; A61K 9/5176; A61K 47/548; A61K 47/55

(57) ABSTRACT

A nanoparticle composition of buckminsterfullerene bonded with epicatechin or a galloyl functional group such as in epigallocatechin gallate, or in tannic acid, is provided that is anti-microbial and efficacious to maintain or re-establish benign healthy cellular homeostasis. In addition, the ability to penetrate hydrophobic malignant microbes via desulfurization is promoted with the addition of phosphonate pendant groups. This further enables the composition to prevent or to treat chronic obstructive pulmonary disorder (COPD), to penetrate fungal spores, and to penetrate the hydrophobic regions of uncontrolled invasive pathological bacteria. The composition can be produced at low temperatures through reactive shear milling. Delivery methods include ingestion, topical application, topical buccal application, inhalation, or injection when used as a medicament or as a food supplement.

### CROSS-REFERENCE TO RELATED APPLICATIONS

This application is a continuation-in-part of International Application PCT/US22/14454 filed on Jan. 28, 2022 which claims the benefit of International Application PCT/US22/12369 filed on Jan. 13, 2022 and the benefit of U.S. provisional patent application 63/161,310 filed on Mar. 15, 2021, all three of which are incorporated herein by reference in their entireties.

### BACKGROUND

### 1. Field of Invention

The present invention is an anti-microbial composition of buckminsterfullerene with one or more galloyl functional groups, where these can include a quinic acid ester (catechin) or a benzo-hydropyran of at least one of a tri-hydroxyphenyl group or a di-hydroxyphenyl group and may also include disodium phosphonate groups. One formulation of this composition is in topical beauty products to mitigate the cellular effects of skin aging and chronic infection by skin bacterial and fungal spores. Another formulation of this composition combats periodontal disease, especially in the case of antibiotic resistant esophageal bacteria. The utility of this and other uses simultaneously and synergistically is to prevent or help treat uncontrolled microbial proliferation, and respiratory ailments such as bronchitis and chronic obstructive pulmonary disease (COPD). The provided delivery methods include ingestion, topical application, buccal application, inhalation, or injection. The composition can be formulated as a prophylactic medicament or used as a food supplement.

### 2. Background Art

Green tea is recognized as the most effective cancer prevention drink in the world. Many or most cancers are microbially induced, such as by a chronic virus, or fungal infection. The cancer and antimicrobial protection properties of various tea extracts are widely known and are clearly linked to a group of polyphenols of a type of flavanol derivatives, or flavonoids. These flavonoids have a chemical structure known as a flavan-3-ol that each possess a 2-phenyl-3,4-dihydro-2H-chromen-3-ol skeleton. Of these flavan-3-ols, there are four highly similar, structurally related catechin molecules that have been identified in green tea as being most efficacious. These catechins are EC (epicatechin), epicatechin gallate (ECG), epigallocatechin (EGC), and epigallocatechin gallate (EGCG). The similarity in both structure and nomenclature can sometimes lead to some confusion. In comparison medical studies, however, EGCG has been demonstrated to provide the most effective and prominent antimicrobial properties of each of these catechin variations, perhaps because the related less molecular weight substances are both more reactive and less thermally stable.

A different class of molecules also having benzo hydropyran groups are the tannins, which have at least one central glucose molecule as their distinguishing characteristic feature. Once benzene is derivatized with hydroxyl groups (OH) these groups are also known as phenyl groups. There are many types of tannins, and their structures may vary widely in the number of di-phenyl benzene and tri-phenyl benzene groups. Many of the tannins can become too large to be of effective use because of poor solubility, especially when multiple glucose groups become entrained into their structures. Of the family of tannins, one molecular member, a very simple one, called tannic acid or penta-m-digalloyl-glucose, has been found to be both low enough in molecular weight and sufficiently reproducible in structure to allow medical evaluation for anti-viral, anti-fungal, and anti-bacterial properties. To date, medical studies and reports indicate that tannic acid is reproducibly about equal in efficacy to EGCG, which points to both EGCG and tannic acid as ideal candidates for future development to enhance their anti-microbial properties. It is thought that the anti-microbial properties in both EGCG and tannic acid arises because they express di-hydroxy phenyl or tri-hydroxy phenyl functionality in their outer molecular structures.

EGCG has a maximum solubility at room temperature of 92 grams per liter of water. EGCG from green tea can induce apoptosis in targeted bacterial and fungal cells. There is at most about 1.25 grams per liter of EGCG in brewed green tea. Epidemiological studies indicate that a minimum of from 2.5 to 3 grams of EGCG ingested per day yields a blood serum concentration of 0.1-1 μM EGCG.

Several studies indicate that EGCG polyphenol confers beneficial effects against several (chronic) microbial pathologies associated with oxidative stress damage to cells, including multiple types of cardiovascular disease, and neurodegenerative diseases which have bacterial, fungal, and viral vectors. In addition, EGCG provides significant antibacterial and antiviral properties that find use in Alzheimer's disease. Reference is made to Sneideris et al., “The Environment Is a Key Factor in Determining the Anti-Amyloid Efficacy of EGCG,” Biomolecules, 2019, 9, 855, and to Yang et al., “EGCG-mediated Protection of the Membrane Disruption and Cytotoxicity Caused by the ‘Active Oligomer’ of alpha-Synuclein,” Scientific Reports, 2017, 7, 17945, showing that EGCG inhibits growth of beta amyloid plaques associated with Alzheimer's disease as well as plaques of alpha synuclein associated with Parkinson's disease. Further, Mahler et al. (2020) “Neuroprotective Properties of Green Tea (Camellia sinensis) in Parkinson's Disease: A Review,” Molecules, 2020, 25, 3926 show a similar effect of EGCG against Parkinson's disease by modulating several gene expressions and acting on mitochondrial REDOX signaling pathways.

EGCG is stable during transit through the intestines when it is taken orally. After it is absorbed by the intestines, EGCG becomes metabolized by intestinal cells. Digested catechins such as EC and EGCG tend to produce glucuronic acids and then may also become partially methylated, forming 3-O-methyl groups. What this means is that oxidized catechins are an attractor for methylating processes and will remove and chemically incorporate the methyl groups from methylating agents. DNA methylation arising from the somatic response to microbial infection is strongly implicated in one of the causes leading to the development of cancers. The oxidative methylation metabolites of the polyphenolic catechins mitigate microbially induced methylation dysfunctions that are often associated with the randomization of the epigenome and therefore the aging process. Incompletely destroyed infective agents are only one of the many possible cumulative errors that can lead to somatic as well as neurological disease states. It is important to note that methylation antagonists are one area of scientific medical research. Therefore, it is widely appreciated that the medical usefulness of the catechins and of EGCG requires more focus and innovation, and that infectious disease prevention is likely not the only medical purpose to which this material or its derivatives will find new application in the future, as the association of EGCG in combination with other molecules has been found synergistic with combating cognitive diseases and is now gaining considerable attention.

The excellent water solubility of EGCG at 92 grams per liter however limits the ability of this polyphenolic molecule to penetrate across lipid cellular membranes and into hydrophobic cellular compartments. This substantially explains the consistently high serving size or dosages of EGCG required to obtain significant health benefits.

An acidic binding substance must be able to penetrate cell lipid membranes to render protection against oxidative and nitrative stress. Some of the current therapeutic strategies have put emphasis on the design of multiple functional properties into molecules or particles that enable them to target enzymes or receptors to help correct the dysfunctions leading to disease states.

While there is great promise in the beneficial effects of EGCG, there are no known strategies to enhance its poor stability against rapid metabolic breakdown to deliver this substance. Attempts to perform encapsulation within microspheres or nanoparticles or attempts to perform a molecular dispersion in polymer matrices to be used as carriers, have proven effective at delivery but remain insufficiently stable.

In all cases, no present state of the art has considered to allow for the possibility of oxidative and reductive cycling of epigallocatechin gallate by tethering it to a specially designed reduction-oxidation (REDOX) center that is capable of both storing and releasing electrons and hydrogen protons for the purpose of recycling the active carboxyl groups of epigallocatechin gallate as an aid to the long-term maintenance of cellular redox homeostasis. Also, there are no present formulations that are clearly designed to ensure that the delivered, released, or free epigallocatechin gallate will be able to penetrate to and then remain at the active molecular signaling and biochemical redox site of the membranes of cell organelles. Part of the reason for this may be those current formulations use targeting that does not adequately provide simultaneous electrostatic and hydrophobic anchoring of epigallocatechin gallate to cellular membranes. Another reason for this may be the lack of a clearly defined function or mechanism to penetrate hydrophobic proteins, especially those containing sulfur-sulfur bridges that confer significant parasitic bacterial resistance.

A significant limitation to the use of dietary EGCG is in the lack of maturity of cell signaling designs. Such design failures are attributed to an incomplete understanding of cell signaling functions and protein messaging effects to defend the cell. Cell signal interactions begin with surface charges at membranes. Surface charges are in contact with the cell cytosol, proteins, DNA, and the lipid membranes of the cell. Some signaling regions, such as at the site of endoplasmic and sarcoplasmic reticula of mitochondria, may become insufficiently engaged in oxidation that is associated with the development of waxy, hydrophobic proteins networked with excess glutathione and other sulfur containing molecules. This REDOX deficit is thought to contribute to dysfunction of the electron transfer cycle that allows proper cellular respiration to take place, and the result can be programmed cell death by apoptosis.

What is therefore needed is a novel therapeutic strategy or unique material used to confer cellular protection and prevent, mitigate, or reverse toxic pathology arising from bacterial and viral induced dysfunction before irreversible damage progresses. Desirably, such a treatment should include a means to remove sources of free radicals even under reducing conditions, to include a very localized and very targeted acidic functionality while also retaining lipid membrane permeation ability. It is believed the present invention provides the first broadly effective discovery of such a composition, having a biological and electrochemical design to confer multiple therapeutic and prophylactic functions to highly targeted protein structures. This composition will change our perspective on applications to boost resistance to the effects of invasive disease pathologies. The use of different carrier formulations enables appropriate methods of administration for this novel composition.

### SUMMARY OF THE INVENTION

This invention is a composition of unique fullerene nanoparticles made from commercially available buckminsterfullerene, optionally including groups of disodium phosphonate (FDSP), on reaction with epicatechin or a galloyl such as penta-m-digalloyl-glucose (tannic acid) or a catechin such as epigallocatechin gallate (EGCG) which may also have a galloyl group.

It is an object to provide a compound that increases the bioavailability, in particular the solubility in aqueous solutions, via the penetrability of hydrophobic regions, and the biological redox functionality of catechin polyphenols such as epigallocatechin gallate having the formula C₂₂H₁₈O₁₁.

The present invention provides the first broadly effective discovery of a compound, having a biological and electrochemical design to confer multiple therapeutic and prophylactic functions. The described carrier formulations, derivatives and compositions enable appropriate methods of administration and their use as a medicament, for example.

Some embodiments of this invention provide a cluster of nanoparticles composed with carbon fullerenes, optionally covalently derivatized with phosphonates having oxidation state of three, and a galloyl such as a catechin moiety, being preferably EGCG, in which this substance is pi-carbonyl bonded from at least one carbonyl group (C═O) to the aromatic regions of the fullerene phosphonate. This enables particularly high solubility of the EGCG in aqueous solutions, thereby increasing the bioavailability of the EGCG and its therapeutic effect.

The pendant acid phosphonates are neutralized with cations being preferably sodium to form disodium phosphonate groups being of a surfactant nature and having a viral or fungal protease inhibiting function via the phosphonate sulfurization reaction. The fullerene disodium phosphonate group has the general formula (C60((OP(ONa)₂)5)ₓ—R_{y}, with R is a selected catechin polyphenol or penta-m-digalloyl-glucose, for example. Preferably, R is EGCG.

In a preferred embodiment, y is 1 or 2, such that the derivate of buckminsterfullerene has the formula C60((OP(ONa)₂)₅—C₂₂H₁₈O₁₁ or C60((OP(ONa)₂)₅—(C₂₂H₁₈O₁₁)₂, respectively.

This fullerene derivative possesses properties which reflect the singular free radical scavenging chemical function of fullerenes, the anti-proliferative function of acidic catechin polyphenols, and the protease control function of cationic disodium phosphonates.

These properties allow the composition ingress to penetrate waxy sulfurized proteins, to confer localized chemical quenching of excessive methylation, and to reduce the ubiquitination of p53 anti-tumor proteins critical to reducing and correcting DNA damage.

In particular, the catechin polyphenol group such as EGCG has antimicrobial properties. By increasing the bioavailability of the galloyl group through targeting redox reactions at cell membranes, the buckminsterfullerene derivative enhances or increases the pharmaceutical properties of the catechin polyphenol group. By provision of stored electrons or protons, in some embodiments, the buckminster-fullerene group allows the regenerative oxidation and reduction of EGCG hydroxyl and carboxyl groups to moderate as an intermediary in the multiplicity of biological redox reactions. It is well known that redox reactions tend to take place at cellular membranes and especially at the internal membrane structures of cellular organelles. This latter ability is highly promoted by the presence of the hydrophobic and lipophilic carbon facets of the buckminsterfullerene adduct, which is attracted to and then anchors in cell membrane lipids, as has been well documented in over 30 years of fullerene biochemical studies. The provision of, for instance, EGCG adducts to buckminsterfullerene thereby effectively targets the catechin polyphenol portion of this moiety to those biologically active sites at membrane surfaces where its activity will find the greatest cellular utility in moderating and regulating redox homeostasis where free radicals are most likely to collect and damage the integrity of the cell membrane.

In one aspect, multiple hydroxyl regions of the catechin functional group of FDSP becomes sacrificially methylated. Suitable FDSP catechins for use as a sacrificial methylation molecule and methylation antagonist include epicatechin (EC), epicatechin gallate (ECG), epigallocatechin (EGC), and epigallocatechin gallate (EGCG).

In a related aspect, FDSP-EGCG is utilized as a methylating antagonist, where FDSP-EGCG becomes sacrificially methylated at hydroxyl regions of the catechin functional group to control dysfunctional methylation because of the systemic aging process. This process is enabled by polygalloyl quinic acid ester functional groups, where more functional groups provide a greater number of methylation sinks at the cost of less local reactivity but improved long term stability for the FDSP-catechins. Specifically, the FDSP-EGCG composition protects the epigenome from methylation induced aging by sacrificial methylation of FDSP-EGCG acting as a demethylating agent otherwise known as a methylation antagonist. This action protects the excessive chemical accretion of methyl (—CH3) functional groups on the epigenome to maintain proper gene expression critical for organism function.

In yet another related aspect, FDSP-EGCG helps regulate epigenetic methylation mechanisms including crosstalk between DNA methylation, histone modifications and non-coding RNAs, and the methylation effects on gene expression. Specifically, FDSP-EGCG controls dysregulated methylation responsible for invasive microbial disease progression. The extraction of methyl groups by sacrificial methylation of FDSP-EGCG therefore provides a pathway to avoid microbially induced tumor and cancer cell generation associated with chronic infections, especially for those microbes that have developed resistance against antibiotic, antiviral, or antifungal drugs.

In yet another related aspect, FDSP-EGCG limits cognitive decline in neurological diseases. In Lewy body dementia and Parkinson's disease, levodopa can become methylated, resulting in the loss of function of the neurotransmitter dopamine that is metabolized from levodopa in the glutamate cycle, leading alpha synuclein plaque formation in the substantia nigra portion of the brain. In Alzheimer's disease, excessive methylation is quenched to limit the formation and agglomeration of beta amyloid plaques. The FDSP-EGCG composition provides a demethylation property by sacrificial methylation of a pendant hydroxyphenyl group that is chemically activated by the presence of the C60 fullerene adduct. The extraction of methyl groups by sacrificial methylation by FDSP-EGCG provides protection of neurotransmitters such as dopamine and its precursor levodopa from functional deactivation by methylation.

In yet another aspect, the sacrificial demethylation function of FDSP-EGCG acts to protect functional regions of cellular proteins from methylation.

In another aspect, the FDSP-EGCG composition provides a desulfurization property by sacrificial oxidation of a pendant phosphonate group. Regions of excess sulfur arise from a local excess of hydrophobic sulfur-protein bonds associated with the bacterial cells or the capsids of some viruses that shields them from the native immune system. The extraction of sulfur from cross-linked and waxy protein agglomerates by FDSP-EGCG leads to a unique mode of protection against invasive cell penetration to better allow the natural immune response access to them.

In another aspect, the nanoparticle ensemble amplifies the well-known bacteriostatic effect of EGCG by the bond to C60, especially for those bacteria that are known as “super bugs” because they have evolved a resistance to prescribed antibiotics.

In a related aspect, certain bacteria commonly live on the skin of many people without causing harm. However, these bacteria can cause skin infections or buccal infections if they enter the body through cuts, open wounds, or other breaks in the skin. A clear alternative to prescribed antibiotics for mouth, skin, or gastric infections by pathological strains of antibiotic resistant bacteria is provided. Non-limiting examples of the types of bacteria that can be treated include methicillin-resistant Staphylococcus aureus (MRSA), group ‘A’ Streptococcus (GAS) or “strep” leading to ‘strep throat’, and Impetigo especially as it is most commonly found on the face as ruptured blisters that form a flat, thick, honey-colored (yellowish-brown) crust.

In another aspect, delivery methods are provided. In one aspect of delivery, a nano-aerosolized composition carries the FDSP-EGCG in a carrier fluid dispenser, and the composition in gasified and delivered to the nose, mouth, trachea, and airways of a patient or user.

In another aspect of delivery, the FDSP-EGCG is adsorbed onto the pore structure of a mineral such as zeolite for oral administration and timed release into the intestinal tract wherein a variation of the silicon to aluminum ratio of this mineral, or a variation in the porosity of diatomaceous earth mineral, or like negative charged mineral, provides both a charged surface and different pore sizes and therefore a timed-release function.

In yet another aspect of delivery, the FDSP-EGCG is formulated into a topical cream carrier for application to the skin and the buccal cavity regions.

In yet another aspect of delivery, the FDSP-EGCG is formulated into an oral solution with sweeteners, flavors, and preservatives suitable to formulate a beverage or to be used as an additive to existing beverages such as traditional tea or coffee.

These and other advantages of the present invention will be further understood and appreciated by those skilled in the art by reference to the following written specification, claims, and appended drawings.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### BRIEF DESCRIPTION OF THE DRAWINGS

Preferred embodiments of the invention will now be described, by way of example, with reference to the accompanying drawings, in which:

FIG. 1 is an illustration of the molecular structures of two exemplary polyhydroxyl phenyl containing molecules, EGCG and tannic acid.

FIG. 2 is an illustration of the molecular structures of alternative flavan-3-ols, being additional exemplary catechin gallate raw materials suitable as substitutes for EGCG.

FIG. 3 is an illustration of the molecular structures of commercially available fullerene disodium phosphonates (FDSP).

FIG. 4 is an illustration of one pentagonal reacted portion of the molecular structure of fullerene disodium phosphonate (FDSP).

FIG. 5 is an illustration of the molecular structures for epigallocatechin gallate (EGCG) reaction with buckminsterfullerene (C60).

FIG. 6 is an illustration of the molecular structures for epigallocatechin gallate (EGCG) reaction with fullerene disodium phosphonate (FDSP).

FIG. 7 is an illustration of a desulfurization reaction leading to tumor penetration and complex formation with p53 protein.

FIG. 8 is an illustration of C60-EGCG or FDSP-EGCG packed into the pores of substrates for timed release, such as Transcarpathian zeolite (clinoptilolite) or diatomaceous earth.

FIG. 9 is an illustration of the method of synthesis of FDSP-EGCG formulated with solvents suitable for nano-aerosol administration.

FIG. 10 is an illustration of the method of synthesis of C60-EGCG and FDSP-EGCG formulated with flavors and preservatives suitable for water-based and solid based oral administrations.

FIG. 11 is an illustration of the method of creating FDSP-EGCG formulated with perfumes and thickeners suitable for topical skin administrations.

FIG. 12 is an illustration of personal administration of aspirated nano-aerosol containing the FDSP-EGCG nanoparticles.

FIG. 13 is an illustration of personal topical skin administration of FDSP-EGCG.

FIG. 14 is an illustration of experimental FTIR data for EGCG.

FIG. 15 is an illustration of experimental FTIR data for C60-EGCG.

FIG. 16 is an illustration of experimental FTIR data for C60-tannic acid.

FIG. 17 is an illustration of experimental FTIR data for FDSP.

FIG. 18 is an illustration of experimental FTIR data for FDSP-EGCG.

FIG. 19 is an illustration of experimental FTIR data for zeolite.

FIG. 20 is an illustration of experimental negative mode mass spectrograph data for buckminsterfullerene (C60).

FIG. 21 is an illustration of experimental negative mode mass spectrograph data for fullerene penta-disodium phosphonate (FDSP).

FIG. 22 is an illustration of experimental negative mode mass spectrograph data for C60-EGCG.

FIG. 23 is an illustration of experimental negative mode mass spectrograph data for FDSP-EGCG.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

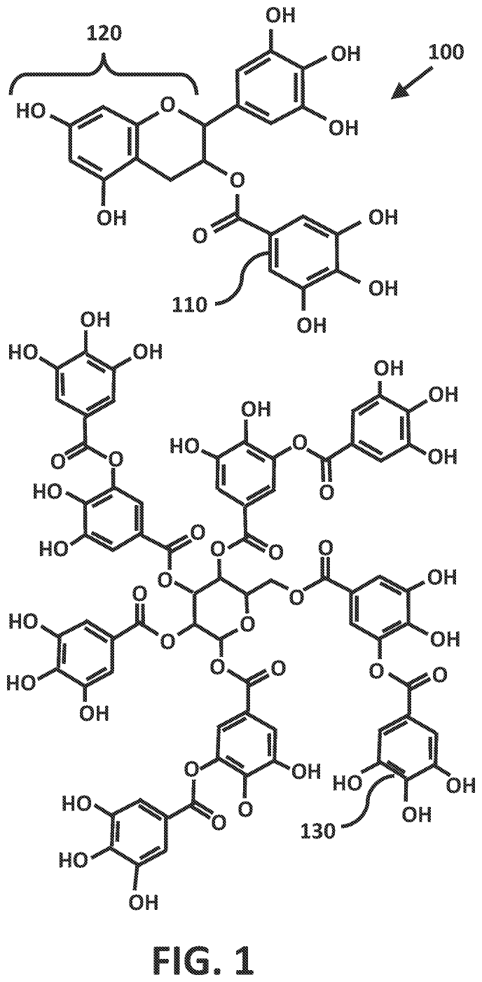

FIG. 1 illustrates two of the most efficacious anti-microbial molecular structures 100 used in the present invention. The molecular structure of epigallocatechin gallate (EGCG) 110 is the most thermally stable and therefore the best catechin used as a raw material to make the derivatives and formulations for the present invention, having chemical formula C₂₂H₁₈O₁₁. The bracketed region 120 of EGCG 110 is more generally known as the chromane or benzo dihydropyran functional group and is a common feature of the acceptable catechin polyphenols in this composition.

An acceptable replacement for EGCG 110 is the polygalloyl quinic acid esters with the number of galloyl moieties per molecule ranging from 2 up to 12, is represented by the tannin molecular structure 130 for tannic acid, also known as penta-m-digalloyl-glucose. Tannic acid is reproducibly about equal in anti-proliferative and anti-bacterial function to the efficacy of EGCG, which points to both EGCG 110 and tannic acid 130 as ideal candidates for development as functional groups to enhance their anti-microbial properties because of each expresses di-hydroxy phenyl or tri-hydroxy phenyl (more generally, polygalloyl) functionality in their outer molecular structures. Substances 110, 130 are used to help create, process, or deliver parts of the composition or their metabolites according to these teachings.

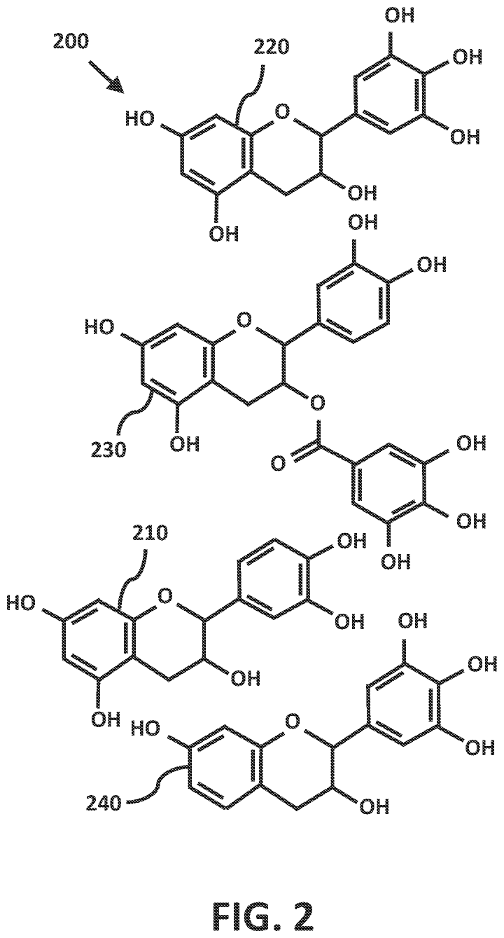

FIG. 2 is an illustration of the molecular structures of flavan-3-ols being exemplary catechins 200 that are acceptable substitutes for EGCG and may be independently selected from the flavan-3-ol group also known as catechins, or physiological metabolites thereof, to be reacted to produce the composition of the present invention. These benzo-hydropyran moieties provide at least one of a tri-hydroxyphenyl group or a di-hydroxyphenyl group of the composition. In some cases, it may be economically desirable to replace the EGCG catechin with an alternative thermally less stable benzo-hydropyran, such as the catechin having formula C₁₅H₁₄O₆ (EC) 210, or the catechin having formula C₁₅H₁₄O₇ (EGC) 220, or the catechin having formula C₂₂H₁₈O₁₀ (ECG) 230, or catechin formula C₁₅H₁₄O₆ (Robinetinidol) 240. Of these alternative selections, the three molecules 220, 230, 240 are experimentally well known to be more reactive polyphenols and can be used to increase the potency of the composition to make it more antimicrobial for some methods of use, such as for treating periodontal disease, than the di-hydroxylated catechin 210. In all cases, the catechin properties become greater and their chemical reactivities become significantly magnified then they are reacted as adducts to the fullerene disodium phosphonates (FDSP), according to the teachings of the present invention.

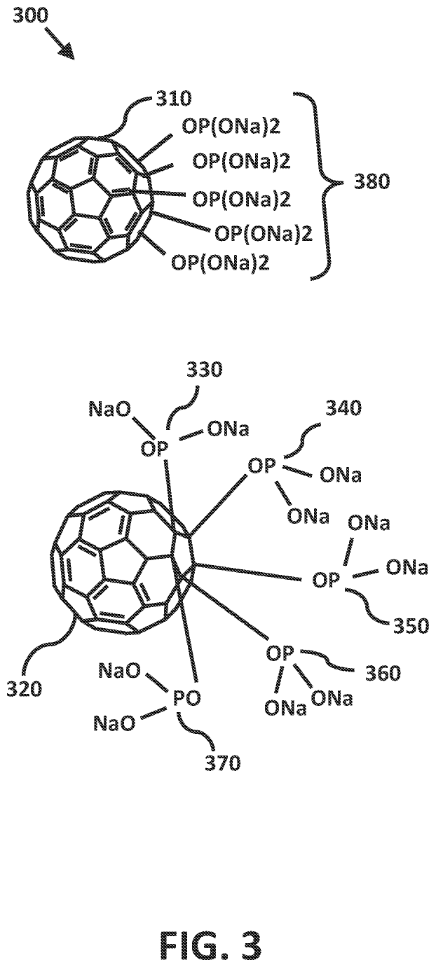

FIG. 3 illustrates two alternative side views of the molecular structures for fullerene disodium phosphonates (FDSP) 300. The commercial product FDSP is a core C60 fullerene 310, 320 that is covalently bonded with five phosphonate groups 330, 340, 350, 360, 370, which can also be illustrated collectively as shown by the bracketed region 380. The FDSP used as a reactant to produce the FDSP catechin composition(s) of this invention can be obtained commercially.

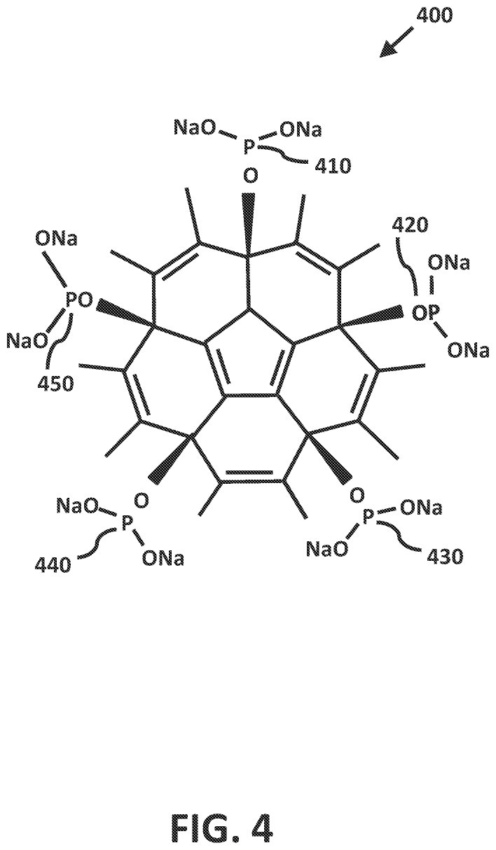

FIG. 4 illustrates a portion of a front view of a central pentagonal carbon region of the buckminsterfullerene (C60) molecular structure of FDSP 400. Five functional groups of disodium phosphonates 410, 420, 430, 440, 450 are covalently bonded proximal to a strained pentagonal region of the C60 molecular cage of FDSP. This substance is commercially available and is used as a reactive reagent to create the galloyl catechin derivatives of the present invention.

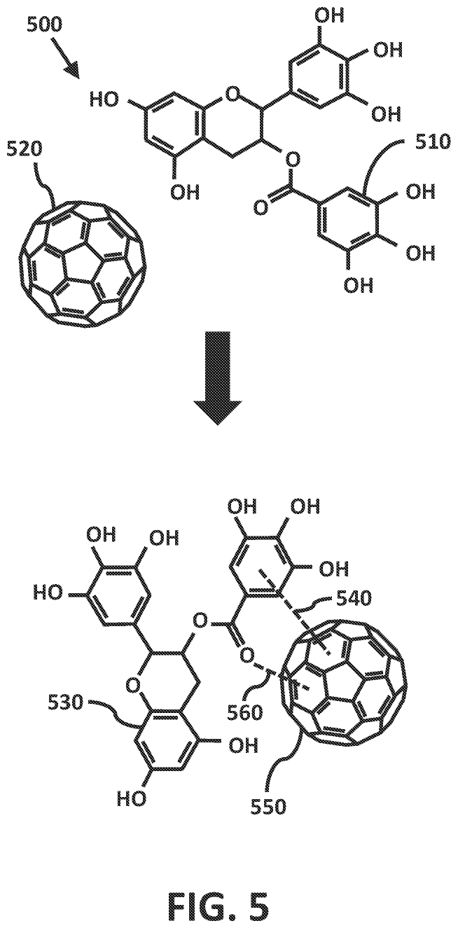

FIG. 5 illustrates the molecular structures for the epigallocatechin gallate (EGCG) reaction with buckminsterfullerene (C60) 500. The exemplary catechin, epigallocatechin gallate (EGCG) 510 is combined to react with buckminsterfullerene (C60) 520 in a reactive shear mill under conditions of shear mixing and pressure. A shear pressure of about 20 grams per square micron is sufficient to create a slightly geometric oblate spheroid of the C60 and simultaneously shift the density of states of the electrons of the carbon cage into anisotropic electrostatic distributions. These electrostatic charges then achieve a metastable state when abutted proximal to simultaneously induced opposing electrostatic charges with at least one polygalloyl catechin, EGCG. This forms the product composition of C60-EGCG provided with pi-carbonyl, pi-cation, and hydrogen bonding where this reaction proceeds in the direction of the large black arrow. At least one aromatic region of the EGCG molecular structure 530 may form a pi-carbonyl bond 540 with the C60 group 550 to significantly stabilize the resulting molecular ensemble. Any carbonyl functional group of EGCG can create a pi-carbonyl bond 560 with an aromatic region of the C60 to further stabilize the C60-EGCG molecular structure. It is to be understood that any combination of the catechin gallates specified herein are acceptable substitutions for the EGCG molecules in the reaction with C60, according to the teachings of the present invention.

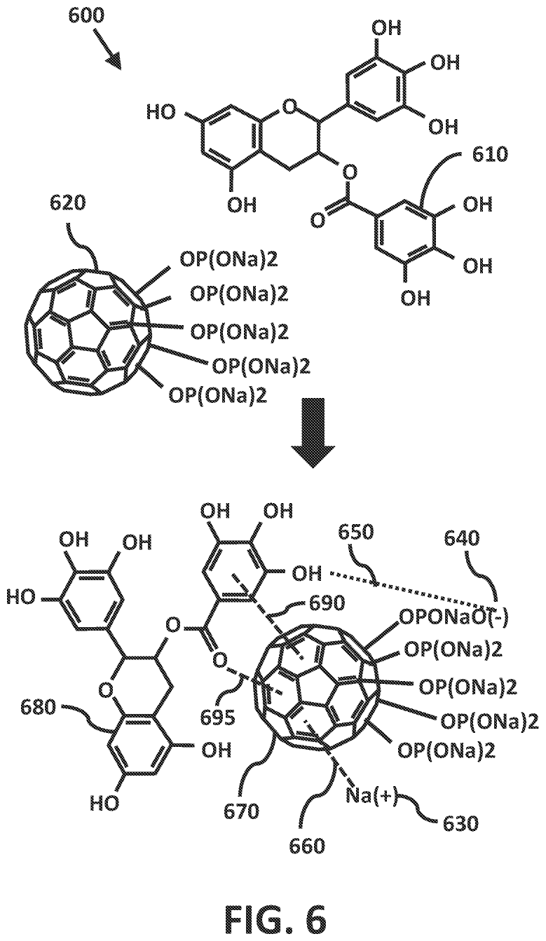

FIG. 6 illustrates a chemical reaction to form a C60-EGCG 600. The exemplary catechin, epigallocatechin gallate (EGCG) 610 is combined to react with fullerene disodium phosphonate (FDSP) 620 in a reactive shear mill under conditions of shear mixing and pressure. A shear pressure of about 20 grams per square micron is sufficient to create a slightly geometric oblate spheroid of the C60 of the FDSP and simultaneously shift the density of states of the electrons of the carbon cage into anisotropic electrostatic distributions. These electrostatic charges then achieve a metastable state when abutted proximal to simultaneously induced opposing electrostatic charges with at least one catechin gallate, EGCG. This forms the product composition of FDSP-EGCG provided with pi-carbonyl, pi-cation, and hydrogen bonding where this reaction proceeds in the direction of the large black arrow. FDSP-EGCG contains a multiplicity of diphosphonate functional groups that may disassociate at least one migrating sodium ion 630 to leave at least one corresponding pendant negative charged anionic phosphonate 640 capable of forming a hydrogen bond 650, illustrated as a dotted line, with any hydroxyl group of the EGCG structural region. The cationic sodium is then able to form a pi-cation bond 660 with any aromatic region of the core C60 functional group 670 of EGCG-FDSP. At least one aromatic region of the EGCG molecular structure 680 may form a pi-carbonyl bond 690 with the C60 functional group to significantly stabilize the resulting molecular ensemble. Any carbonyl functional group of EGCG can create a pi-carbonyl bond 695 with an aromatic region of the C60 functional group to further stabilize the EGCG-FDSP molecular structure. It is to be understood that any combination of the catechin gallates specified herein are acceptable substitutions for the EGCG molecules in the reaction with FDSP, according to the teachings of the present invention.

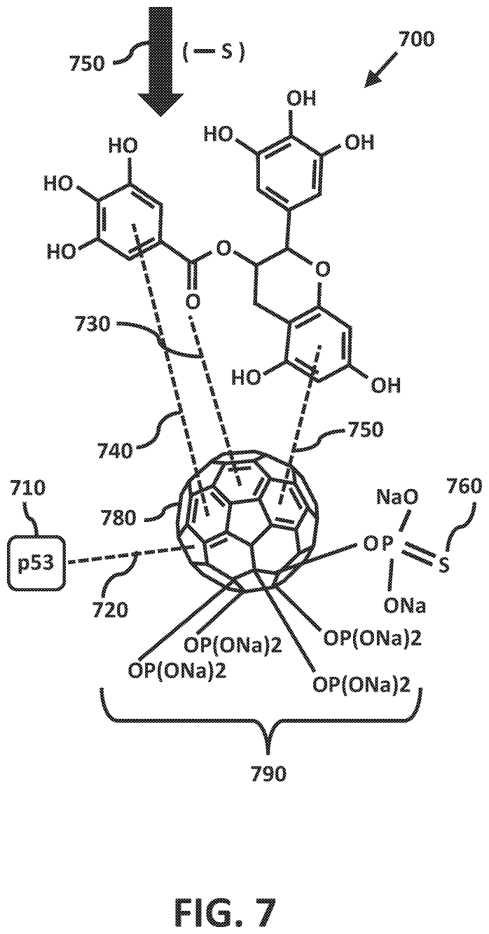

FIG. 7 illustrates a desulfurization reaction of FDSP-EGCG 700, leading to bacterial and viral capsid penetration. The bacterium or virion is represented herein as a schematic geometric form 710. A pi-bond to the bacterial or viral protein 720 shown by a dashed line, isolates and immobilizes the invasive particle or cell. The EGCG is provided with aromatic pi to carbonyl bonds 730, and with aromatic pi to aromatic pi stacking bonds 740, 750 to the C60 functional group as shown by dashed lines in this molecular structure.

The presence of phosphonate groups of oxidation state three is provided in the molecular structure of FDSP-EGCG to penetrate the sulfur-rich hydrophobic region of a bacterium or virion, as well as to desulfurize fungal proteases associated with respiratory pathology such as valley fever, or to desulfurize viral proteases. The phosphonate sulfurization reaction proceeds by extraction of sulfur (S) as indicated by the black arrow 750, where the source of extracted sulfur can be a local excess of glutathione and sulfur-protein bonds associated with the waxy region that separates tumor cells from the native immune system carried by aqueous phase physiological plasma such as blood in the circulatory system. One of the phosphonate groups of FDSP-EGCG is sulfurized by the acquisition of a sulfur atom 760. The sulfurization results in a sulfurized phosphonate having phosphorus of oxidation state 5. Sulfurization demonstrates the superiority of FDSP-EGCG over C60-EGCG in penetrating the regions masked by sulfur bonded protein regions.

The remaining four disodium phosphonate groups 790 may continue to act as desulfurization agents, as these can provide additional subsequent desulfurization reactions, thereby enabling proteins complexed with FDSP-EGCG to penetrate even more deeply into the waxy sulfurized coatings around invasive virions, fungal spores, and pathogenic bacteria cells, according to the teachings of the present invention.

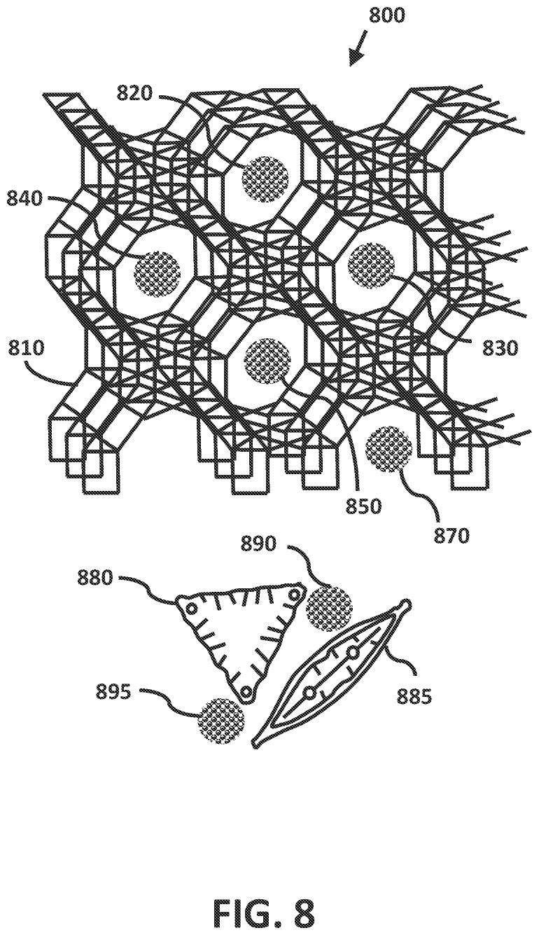

FIG. 8 illustrates the porous substrate zeolite or diatomaceous earth impregnated with C60-EGCG or FDSP-EGCG 800. Transcarpathian zeolite (clinoptilolite) 810 is a type of mineral provided with a highly negatively charged network structure achieving a system of reproducible and well-defined pores and channels. Clinoptilolite zeolite is well known to adsorb nitrogen containing compounds including ammonia, amino acids, and other positive charged molecules. Similarly, Clinoptilolite zeolite is optionally used herein to adsorb thiamine (vitamin B1) as positive counter-ion and hydrogen bonding adduct. The thiamine adducts can be used to stabilize the impregnation with the composition of FDSP-EGCG in the form of a multiplicity of clusters 820, 830, 840, 850, 860, and 870 having cluster sizes sufficiently small to fit within the pore regions, being greater than 100 nanometers and less than about 5 microns in size. It is also known that at pH greater than 7, as well as under saline or physiological ionic salt conditions, clinoptilolite zeolite displaces and expresses the positively charged nitrogen compounds and counterions stored within the pores. The salt and pH moderated regenerant property of clinoptilolite towards reversible expression and release of positively charged nitrogen compounds has led to the widespread economic commercial adoption of clinoptilolite Transcarpathian zeolite as a dietary supplement.

Diatomaceous earth is a silicate bearing mineral composed of a multiplicity of silicon dioxide skeletons of diatoms having a multitude of shapes 880, 885 and being from 30 to about 200 nanometers in size. The negatively charged diatomaceous silicates can adsorb thiamine (vitamin B1) as a positive counter-ion and hydrogen bonding adduct; these adducts stabilize the impregnation with the composition of FDSP-EGCG in the form of a multiplicity of clusters 890, 895. At pH greater than 7, as well as under saline or physiological ionic salt conditions, diatomaceous earth slowly diffusion releases and expresses FDSP-EGCG and thiamine counterions stored within the pores and the spaces between the silicate structures of the diatoms to achieve a timed-release of the FDSP-EGCG composition into the digestive tract.

The counter ion-exchange property of Transcarpathian zeolite (clinoptilolite) or diatomaceous earth or other solid pharmaceutical grade minerals may be used as adjuvant delivery or timed-release delivery in any combination whatsoever, to perform timed digestive release of the composition of the present invention as one method of oral delivery of the composition of the present invention, according to these teachings.

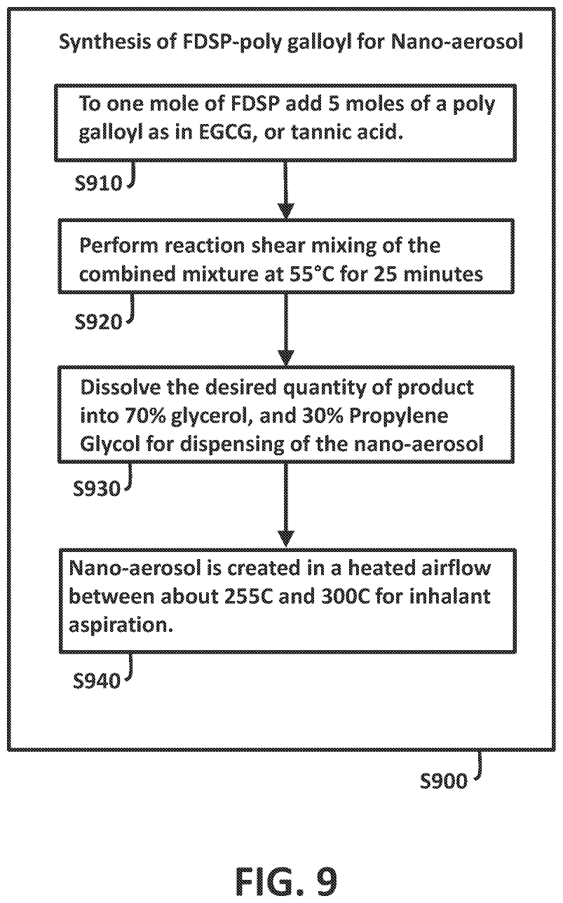

FIG. 9 is a flowchart representation of an exemplary scalable synthesis method S900 of nano-aerosol FDSP-EGCG formulated for nano-aerosol administration. In step S910 To one mole of FDSP, add 5 moles of a catechin; EGCG is the preferred catechin. In step S920 the prepared dry powder mixture is reaction shear milled at about 55° C. to achieve the desired FDSP-EGCG product whereby a shear pressure of about 20 grams per square micron is sufficient to create a slightly geometric oblate spheroid of the C60 of the FDSP and simultaneously shift the density of states of the electrons of this cage molecule into anisotropic electrostatic distributions that achieve a metastable state when abutted to simultaneously induced opposing electrostatic charges with least one abutting proximal EGCG polyphenol. In step S930, the desired concentration of FDSP-EGCG is created by dissolving a weighed amount of the dry powder into a 70% glycerol and 30% polypropylene glycol solvent mixture by volume. In step S940, a metered amount of the nano aerosol fluid from step S930 is generated by a commercially available electronic dispensing device suitable for client inhalant aspiration by means of a heated airflow between about 255° C. and 300° C. to create the nano-aerosol, according to the teachings of the present invention.

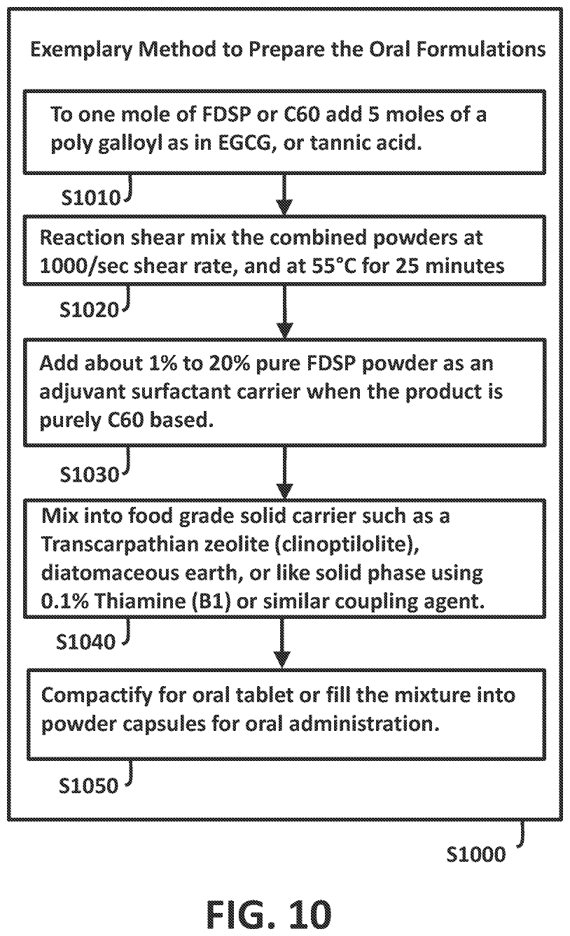

FIG. 10 is a flowchart representation of an exemplary scalable method S1000 for synthesis of oral administered FDSP-galloyl. In step S1010 about 1 mole of commercially available FDSP is combined with nominal 5 moles of a catechin, EGCG or tannic acid. In step S1020 the combined mixtures are milled at about 1000/sec shear rate and about 55° C. to achieve the desired FDSP-EGCG reaction product. In this process, a shear pressure of about 20 grams per square micron is sufficient to create a slightly geometric oblate spheroid of the C60 of the FDSP and simultaneously shift the density of states of the electrons of this cage molecule into anisotropic electrostatic distributions that achieve a metastable state when abutted to simultaneously induced opposing electrostatic charges with least one abutting proximal EGCG polyphenol. In step 1030, the FDSP-EGCG from step 1020 is mixed into food grade slow-release solid carrier material such as a Transcarpathian zeolite (clinoptilolite), diatomaceous earth, or like porous solid phase. This operation is substantially enabled by the introduction of a vitamin B1 additive to assist with creating a positive counter-ion charge coupling with both the negative charges in the porous solid phase and the negative charges in the FDSP-EGCG. This process can be performed in a standard industrial kneading device such as food processing mixers that are typically used for making bread. In step S1040, the desired concentration of FDSP-EGCG is created by dissolving a weighed amount of the dry powder mixture with the porous scaffold component into a mold for pressing into an oral tablet. Alternatively, a weighed dosage of this power mixture is filled into capsules to be administered for oral administration of the time-release formulation. This serving size or dosage may then be dispersed into any amount of water if desired, prior to oral administration, to ease consumption. It is understood that such a water dispersion of the time-release formulation is unstable and subject to settling on standing for periods of greater than a few hours. When desired, this formulation may be dispensed into aqueous media for later distribution at any time for later oral administration, with the provision of optional viscosity modifiers that can be added to this mixture to stabilize the insoluble mineral components from settling therein as a minor variation to this method. This enhances the long-term esthetic appeal of the solid dispersed into an aqueous medium, while simultaneously maintaining the time-release feature of the porous solid insoluble carriers, according to these teachings.

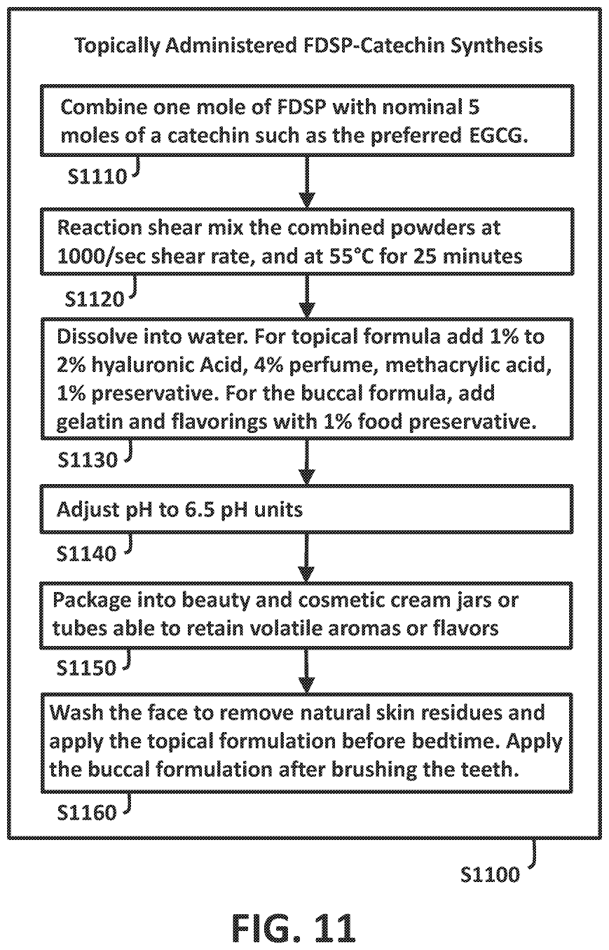

FIG. 11 is a flowchart representation of an exemplary scalable method S1100 for producing and applying a topical skin or buccal administered FDSP-catechins. In step S1110 To one mole of FDSP, add 5 moles of a catechin as in the preferred EGCG. It is to be understood that any of the catechins EC (epicatechin), epicatechin gallate (ECG), or epigallocatechin (EGC) can substitute for epigallocatechin gallate (EGCG). It should be noted that the use of poly-galloyl tannic acid for beauty products should be avoided because it may stain the skin, but when the topical formula is to treat skin infections or esophageal infections, the tannic acid forms a substantially greater surface bond to skin and mouth cells for superior antimicrobial function. In step S1120 the mixture of step S1110 is reaction shear milled at about 55° C. to achieve the desired FDSP reaction product. In this process, a shear pressure of about 20 grams per square micron is sufficient to create a slightly geometric oblate spheroid of the 6C0 of the FDSP and simultaneously shift the density of states of the electrons of this cage molecule into anisotropic electrostatic distributions that achieve a metastable state when abutted to simultaneously induced opposing electrostatic charges with least one abutting proximal catechin polyphenol such as EGCG. In step S1130 the product of Step 1120 is dissolved into water. For topical skin formulations, about 1% to 2% of the product is added to hyaluronic acid, along with about 4% perfume, a desired amount of methacrylic acid for viscosity enhancement, and 1% preservative. However, for buccal solutions, a commercially available gelatin and desired flavors can be used along with 1% sodium sorbate as a food preservative. In step S1140, the pH of the acidic FDSP-EGCG composition is adjusted to prevent mold or bacterial growth with an acceptable range of 5 to 6.7 and a nominal value of 6.5 by neutralization with sodium hydroxide (NaOH) with adequate mixing to ensure a uniform cream or lotion. In step S1150, this material composition is transferred into beauty and cosmetic cream jars or tubes having a sufficiently hermetic seal to able to retain the volatile aromas or flavorings. In step 1160, the face is washed to remove natural skin residues prior to applying the topical formulation, such as before bedtime. The buccal formulation can be applied, for example, after brushing the teeth.

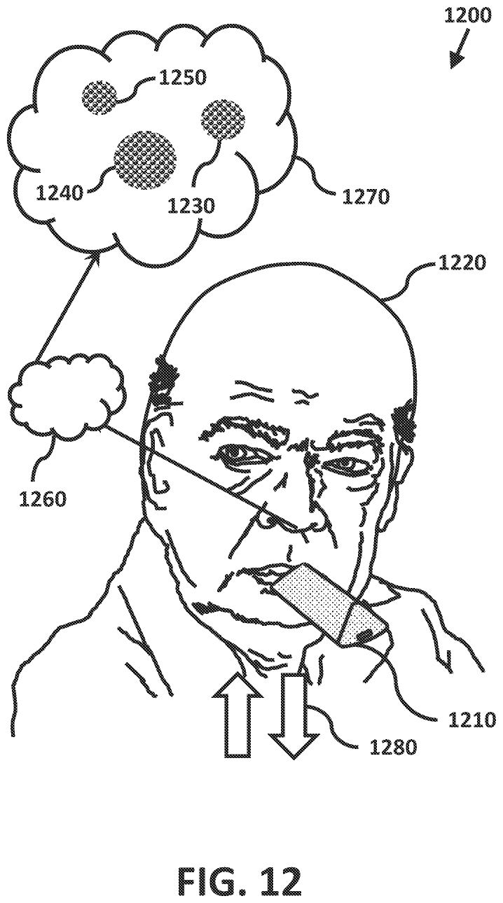

FIG. 12 illustrates a method S1200 for the personal administration of aspirated nano-aerosol delivery of fullerene penta-disodium phosphonate epigallocatechin gallate (FDSP-EGCG). The nano-aerosol generating device 1210 filled with FDSP-EGCG dispensing solution is provided for dispersing and nebulizing the inhalant gas including the nano-particles. The device 1210 may also be more commonly known as a nebulizer, or an electronic vaporizing device, or an electronic cigarette, or the functional part of a hookah to be shared among several users. In all cases these systems serve to carry the FDSP-EGCG in a carrier fluid dispenser 1210, and to transfer that composition in nebulized form along with an aerosolized solvent in a substantially gaseous dispersion to the nose, mouth, trachea, and airways of a patient or user 1220. One intended use of the FDSP-EGCG composition is to treat, delay or arrest the incidence of microbial infections wherein the nano-aerosol can expedite targeted delivery to the brain by avoiding a passage through the digestive system.

Some of the nano-aerosolized composition is exhaled and shown as particulate clusters 1230, 1240, 1250 within exhaled smoke puffs 1260 and 1270 emitted on exhalation as indicated by the direction of thin line arrows radiating away from the nose of the subject 1220. Delivery of the C60-FDSP-EGCG nano-aerosol composition from dispenser 1210 provides antioxidant properties to the mucus airway tissues wherein destruction of free radicals and oxidants associated with microbial invasion is provided. Systems that may be used for the method of dispersion of the FDSP-EGCG represented by dispenser 1210, include, without limitation, any of the electronic cigarette devices produced internationally and listed in Appendix 4.1, “Major E-cigarette Manufacturers” of the “2016 Surgeon General's Report: E-Cigarette Use Among Youth and Young Adults” published by the Center for Disease Control and Prevention (CDC), Office of Smoking and Health (OSH) freely available at the CDC.GOV website, or any combination of piezoelectric, resistively heated, or inductively heated vaporized fluid delivery methods that can be utilized to deliver the composition of the present invention, especially when approved as a medical drug delivery device. Each embodied variation of such methods without limit are intended to aspirate aerosols as the method of therapeutic substance delivery of the composition of the present invention directed into the nasal cavities, mouth, tracheal breathing orifice, or intubated trachea of a patient. The supply direction of nebulized feed of FDSP-EGCG on inhalation and exhalation are delivered into the airways and lungs of the intended patient by the flow of supplied air as indicated by the direction of upward and downward facing large white arrows 1280, when used according to these teachings.

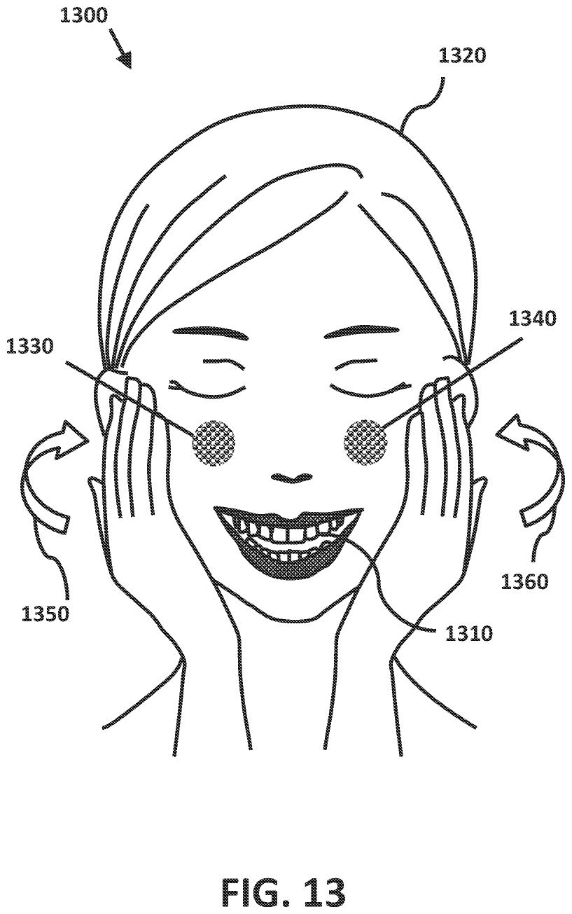

FIG. 13 illustrates personal topical skin administration and buccal administration of fullerene penta-(disodium phosphonate) epigallocatechin gallate (FDSP-EGCG) composition 1300. A semi-liquid slurry dispersion, cream, ointment, or lotion can be used to contain and transfer the administered formula, as a somewhat different formulation is required depending on whether the application is for topical use on the skin or to coat tooth surfaces in the oral or buccal cavity, which is located inside the mouth 1310. The skin care formula can be applied by the user 1320 in regions such as the face 1330, 1340. Application of the skin care formulation can be by means of circular rubbing motions as indicated by the direction of arrows 1350, 1360. The skin-care formulation then confers topical antimicrobial properties such as for MRSA and other antibiotic resistant skin infections, anti-aging and skin brightening functions, and to promote resistance to the onset of skin microbial infections. In the case of the buccal administration to the oral cavity, which is located inside the mouth 1310, the oral mucosal antibacterial functions of the nanoparticle ensemble are to promote anti-gingivitis properties, such as to treat periodontal disease, especially for those bacteria that are known as “super bugs” because they have evolved a resistance to prescribed antibiotics, as well as to promote anti-esophageal fungal treatment properties, as most fungal infections are a co-host for viral infections. Non-limiting examples of the type of bacteria that can be treated include methicillin-resistant Staphylococcus aureus (MRSA), group ‘A’ Streptococcus (GAS) or “strep” leading to ‘strep throat’, and Impetigo especially as commonly found on the face in the form of ruptured blisters that form a flat, thick, honey-colored (yellowish-brown) crust. The formulations are to be synthesized and administered according to the teachings of the present invention.

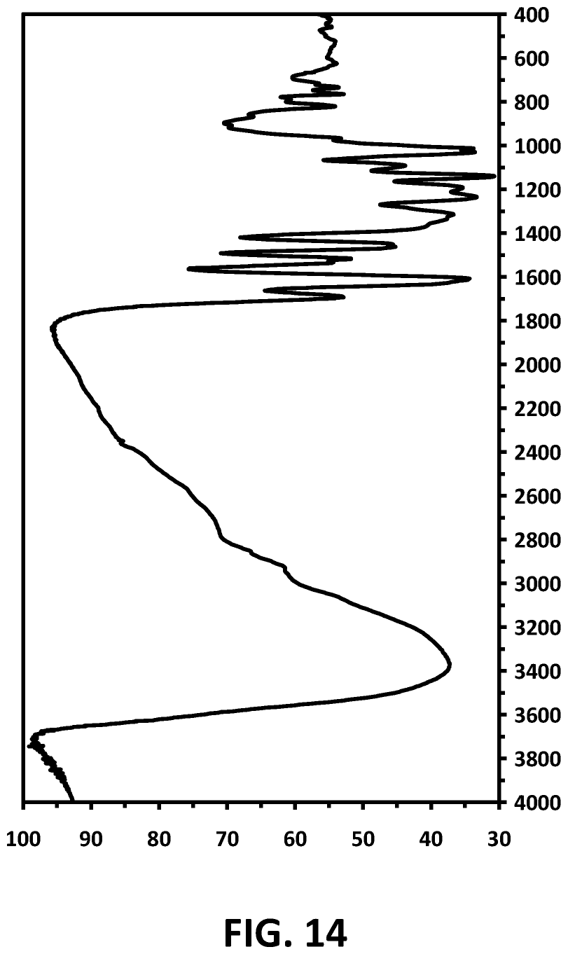

FIG. 14 illustrates experimental FTIR data for EGCG raw material. The sample was prepared by the method of mixing, crushing, and consolidating under 7 metric tons of pressure, about 0.001 grams of analyte with 1 gram of a diluent solid material that is substantially transparent to infrared light, this diluent being anhydrous potassium bromide (KBr), which then flows under pressure to form a translucent pellet of about 0.4 mm thickness. Spectral background subtraction in air using a control pellet of the same mass and thickness having pure KBr was used to obtain a baseline instrument infrared spectral transmission response. This method is generally referred to as the ‘KBr pellet’ sample preparation method, and it is used hereinafter throughout for each FTIR experimental data collection and spectral analysis. The Fourier transform infrared spectrophotometer used herein to obtain FTIR spectra throughout, is a model RF6000 FTIR instrument manufactured by Shimadzu of Japan.

The sample of EGCG analyte prepared by KBr pellet obtains a broad characteristic absorbance from 3100 cm⁻¹ to 3600 cm⁻¹ arising from hydroxyl (OH) functional groups bonded to each aromatic ring. Absorbances at 1609 cm⁻¹, and 1646 cm⁻¹ arise from the carbonyl group (C═O) that links between the trihydroxybenzoate group and the chromane (benzo dihydropyran) ring. The absorbance at 1450 cm⁻¹ arises from the C—H group present in the Chromane ring. Absorbances at 1150 cm⁻¹ and 1091 cm⁻¹ are attributed to the hydroxyl (OH) group, and the peak at 817 cm⁻¹ is attributed to carbon-hydrogen (CH) stretch pendant from the aromatic ring. Comparison of the illustrated experimental FTIR data for EGCG 1800 indicates similarity to the FTIR absorbances reported for EGCG that are generally available from the scientific literature for confirmation of this reactant material when used according to the teachings of the present invention.

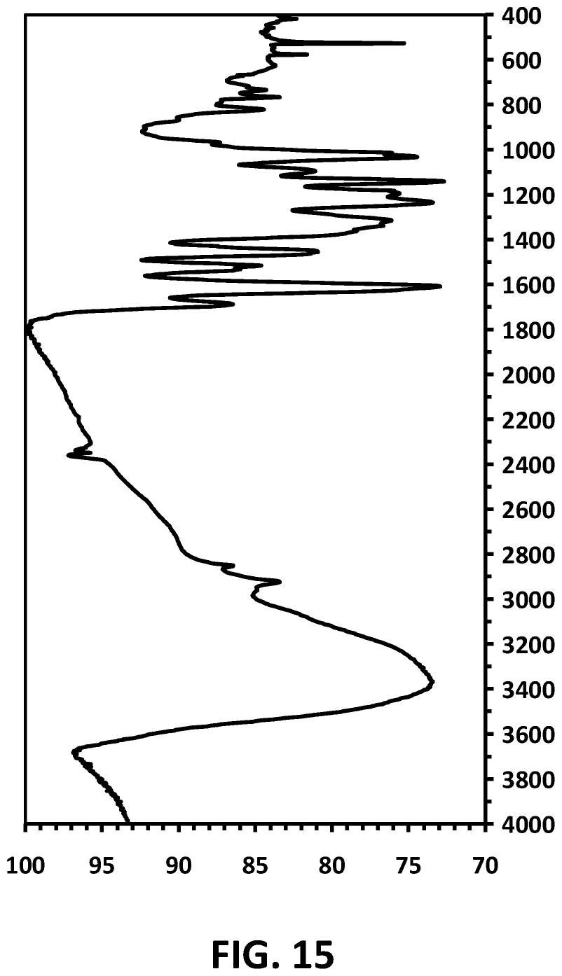

FIG. 15 illustrates experimental FTIR data for C60-EGCG. A very strong and sharp C60 fullerene aromatic carbon-carbon (C—C) stretching band appears at 576 cm⁻¹ and, and a less intense but also sharp carbon-carbon absorbance appears at 526 cm⁻¹. Constrained carbon-hydrogen stretching bands appear at 2921 cm⁻¹ and 2851 cm⁻¹ attributed to the likely interaction of the EGCG ring structures with the fullerene ring structure through aromatic pi bonding. The pure EGCG carbonyl absorbance at 1646 cm⁻¹ shown in FIG. 14 is now seen to be decreased in intensity and shifted to 1684 cm⁻¹; however, the pure EGCG carbonyl absorbance at 1609 cm⁻¹ shown in FIG. 14 is verifiably identical in intensity and remains at 1609 cm⁻¹. These carbonyl characteristics are attributed to a shifted stretching angle of the proximal chromane group through the oxygen bridge next to the carbonyl group and provide evidence for an altered geometry of the EGCG functional group as it wraps around the fullerene group through aromatic pi interactions. This design feature shows that the geometry of the ECGC functional moiety has been altered but the acidity of the hydroxyl groups has been preserved in a manner that favors the chemical interaction of C60-EGCG as a demethylating agent or a sacrificial methyl group sponge. These changes in infrared absorbances are attributed to altered or spatially confined geometry impacting bond mobility in bending and stretching modes associated with the formation of pi-pi bonds according to the teachings of the present invention.

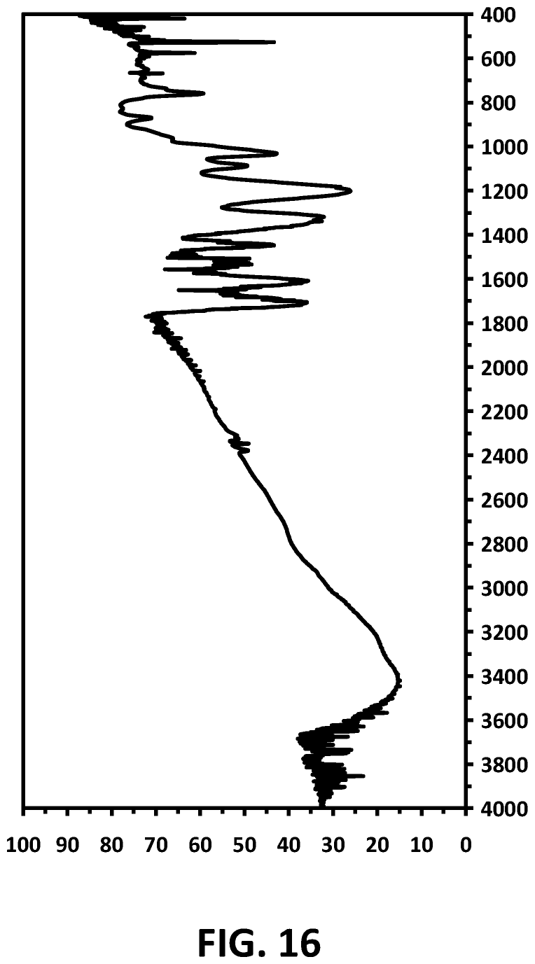

FIG. 16 illustrates experimental FTIR data for C60-tannic acid. Characteristic sharp C60 fullerene aromatic carbon-carbon stretching bands appear at 526 cm⁻¹ and 576 cm⁻¹. Two carbonyl (C═O) bands having different molecular environments appear at 1609 cm⁻¹ and at 1706 cm⁻¹. The central glucose molecule makes strong carbon-oxygen vibrational contributions at 1197 cm⁻¹ and 1318 cm⁻¹. The broad absorbance region from 3680 cm⁻¹ to about 2870 cm⁻¹ is attributed to the hydroxyl functional groups of the poly-galloyl structures in this molecule.

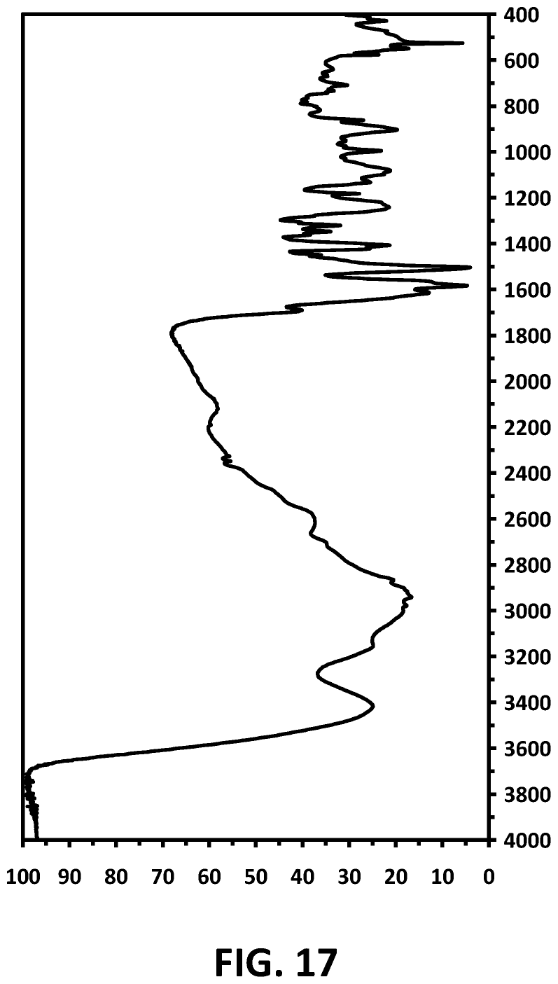

FIG. 17 illustrates experimental FTIR data for fullerene disodium phosphonate (FDSP). A characteristic and very strong and sharp C60 fullerene aromatic carbon-carbon stretching band appears 526 cm⁻¹. The strong and broad absorbance peak ranging from 3250 cm⁻¹ to 3650 cm⁻¹ is attributed to hydroxyl functional groups pendant from the phosphonate adducts which have not been completely neutralized by sodium ions. The absorbance at 1581 cm⁻¹ is attributed to phosphorous-oxygen stretching and are associated with fullerene phosphonates characteristic for purposes of identification when this reactant is used according to the teachings of the present invention.

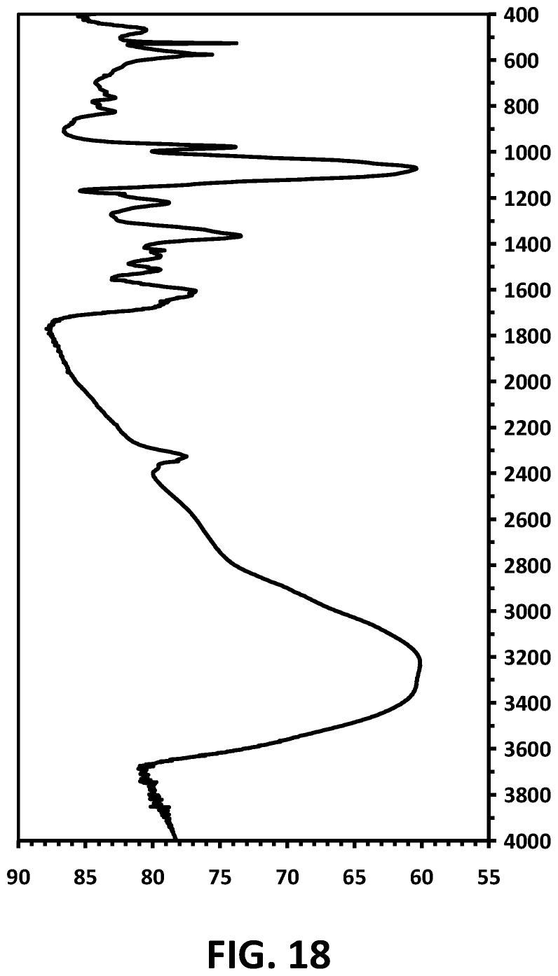

FIG. 18 illustrates experimental FTIR data for fullerene disodium phosphonate-epigallocatechin gallate (FDSP-EGCG). A significant new absorbance at 1073 cm⁻¹ is observed that corresponds with the disappearance of previously observed absorbances at 1150 cm⁻¹ and 1091 cm⁻¹ attributed to the hydroxyl (OH) groups of EGCG as described for FIG. 14. This effect is attributed to the change of the EGCG hydroxyl group stretch from being dissimilar in two regions with respect to planar phenolic symmetry to a uniformly constrained hydroxyl group stretch that is geometrically dominated by the proximal phosphonate groups at their distal ends, and the proximal fullerene with pi-pi bonds appending to each aromatic phenyl group of the EGCG, wherein these combined effects indicate a strongly held and partially wrapped configuration. This design feature shows that both the geometry as well as the acidity of the EGCG functional moiety, according to these teachings.

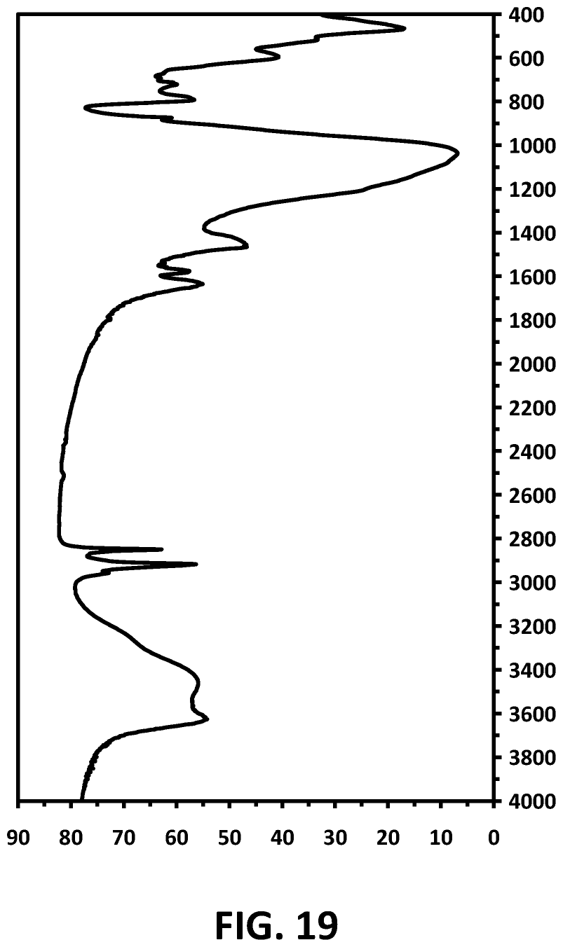

FIG. 19 illustrates experimental FTIR data for the natural Transcarpathian zeolite (clinoptilolite). There are over 40 well known natural zeolites and about 160 synthetic zeolites, where the synthetic zeolites have fine control over pore size as compared with natural zeolites due to their purity and control over the silicon to aluminum ratio; herein it is desirable to have the natural zeolite provided with a wide range of pore sizes to suit the timed-release application. It is to be understood that the use of synthetic zeolites, especially a blend of synthetic zeolites having different pore sizes, are an equivalent and acceptable substitution. The broad absorbance peak observed at 3441 cm⁻¹ is attributed to the existence of the adsorbed water hydroxyl group stretching vibrations (OH). The absorbances at 2918 cm⁻¹ and 2850 cm⁻¹ arise from trace carbon hydrogen stretching of organic materials adsorbed onto the structure of the natural zeolite. The absorbances at 1634 cm⁻¹ and 1574 cm⁻¹ are attributed to two different types of steric environments associated with hydroxyl (OH) bending vibrations. The absorbance peak at 1455 cm⁻¹ is attributed to adsorbed amine contributing to a signal of nitrogen-hydrogen bending (—NH) which correlates with the absorbance at 3626 cm⁻¹ for nitrogen-hydrogen (NH) stretching vibrations. The very intense and broad beak at 1031 cm⁻¹ has a characteristic shoulder absorbance at 1197 cm⁻¹ which collectively characterize the primary absorbance patterns of zeolite arising from its aluminum-oxygen (AlO) and silicon-oxygen (SiO) bending vibrations, where the position of this band depends on the aluminum to silicon ratio and determines the number of the Al atoms per formula unit. The asymmetric stretching due to the internal vibrations of the zeolite silicon-oxygen (SiO) framework tetrahedra occurred at 790 cm⁻¹. The symmetric stretching due to the internal vibrations of silicon-oxygen (SiO) framework tetrahedra is attributed to the absorbance peak at 719 cm⁻¹. The symmetric stretching due to the internal vibrations of negatively charged silicon-oxygen (SiO(—)) framework is attributed to the absorbance peak at 586 cm⁻¹. The strong absorbance at 464 cm⁻¹ is attributed to the bending of the zeolite framework tetrahedra. The experimental FTIR data for this natural Transcarpathian zeolite (clinoptilolite) indicates significant chemical similarity to the FTIR absorbances reported for zeolite that is generally available from the scientific literature and may be used for confirmation of the identity of zeolite absorbent material used to make a timed-release nanoparticle filled catechin formulation for the method of inclusion and carrying of those materials, according to these teachings.

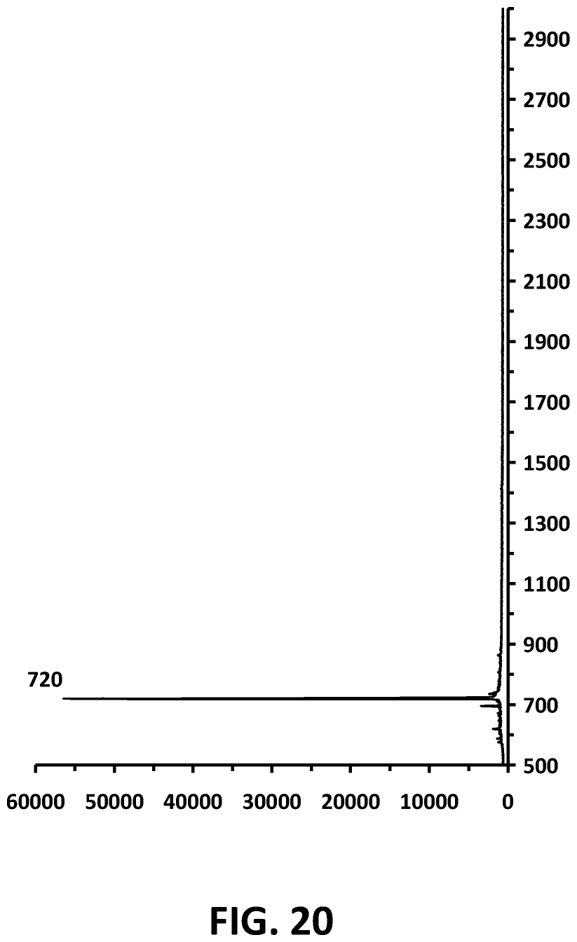

FIG. 20 illustrates experimental negative mode MALDI-TOF mass spectrograph data for Buckminsterfullerene (C60). This sample, as well as each of the subsequent MALDI-TOF experimental test results hereinafter, was introduced for test by laser vaporization into a Voyager Mass Spectrograph from Applied Biosystems (Foster City, Calif., USA). Negative mode bombardment was by fast moving electrons at about 70 eV energy. This resulted in molecular fragmentation and electron removal from the highest molecular orbital energy as molecular ions were formed. The ratio of mass to charge (m/z) is used to determine the molecular ion fragments to help determine the pieces of the original molecule in this assay. The mass peak at 720 m/z corresponds to the molecular ion of fullerene C60. The overall experimental test results characterize the molecular ion breakdown product of buckminsterfullerene (C60), where C60 may be used to further synthesize the composition of the present invention.

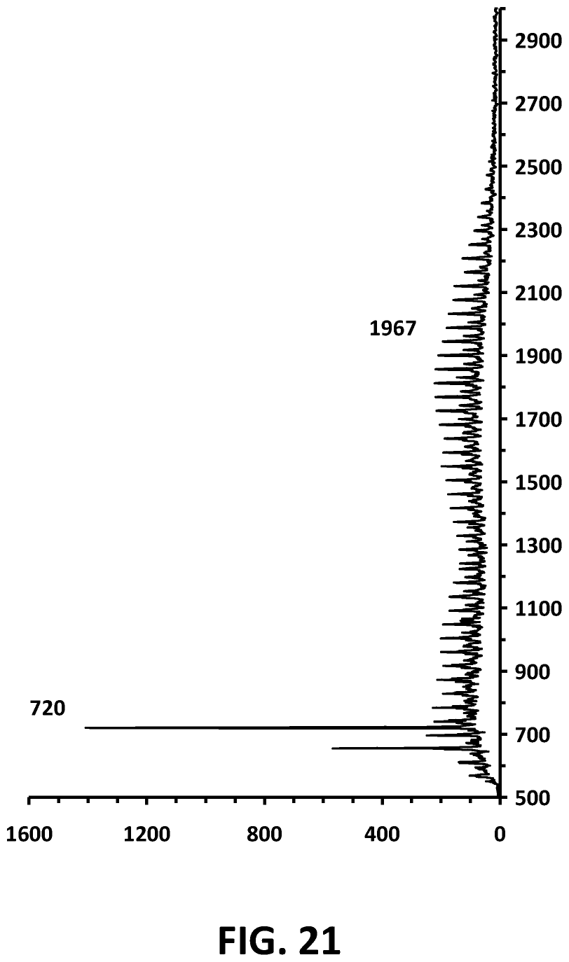

FIG. 21 illustrates experimental negative mode MALDI-TOF mass spectrograph data for FDSP. Negative mode bombardment was by fast moving electrons at about 70 eV energy. The mass peak at 720 m/z corresponds to the molecular ion of fullerene C60 functional group. The large number of sharp peaks with a cluster maximum at about 1967 m/z are attributed to the spallation products of partially ablated disodium phosphonate functional groups. The overall experimental test results characterize the molecular ion breakdown products of FDSP, where FDSP may be purchased commercially or can be synthesized as explained herein and then is to be used to further synthesize the composition of the present invention.

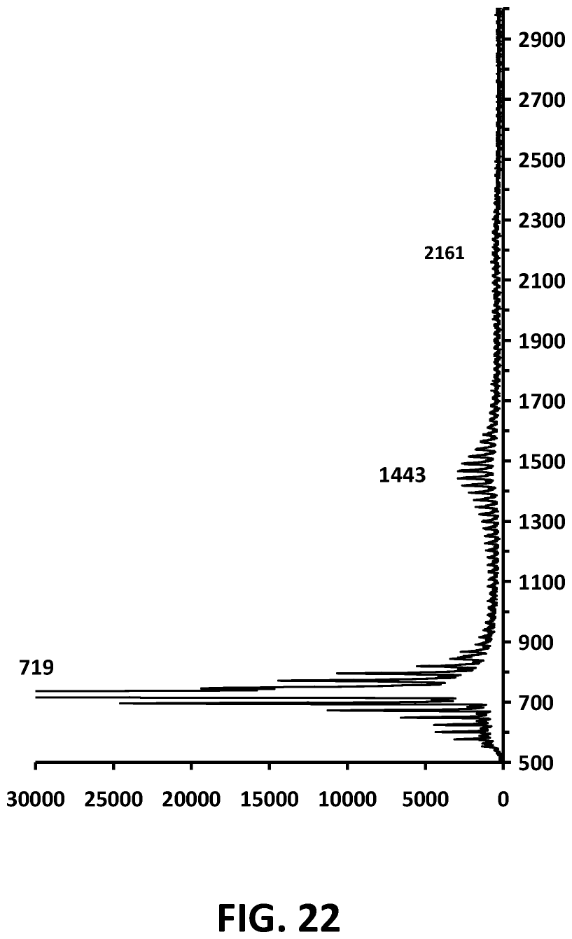

FIG. 22 illustrates experimental negative mode MALDI-TOF mass spectrograph data for C60-EGCG. The appearance of a multiplicity of spikes having separation of mass to charge ratio of 24 are attributed to the loss of dicarbide ions (C—C) associated with the presence of pi-pi aromatic bonds. The primary peak at about 726 mass to charge ratio is attributed to the fullerene functional group of 720 m/z having 6 adducts of hydrogen, and the cluster of peaks at about 1443 m/z are attributed to the presence of non-covalent pi-pi intercalation of EGCG, some of which may be shared between dimeric fullerene functional groups. The minor trace of peaks above this mass may indicate some traces of multimeric fullerene chains with signals below a threshold that is useful for interpretation and analysis. The characteristic mass spallation patterns are consistent with and representative of the formation of C60-EGCG for this component of the composition according to the teachings of the present invention;

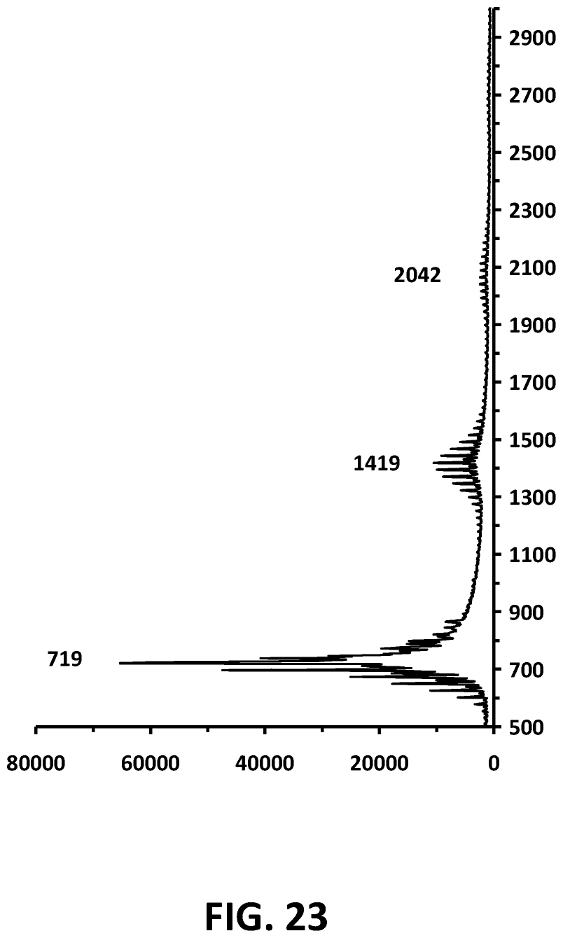

FIG. 23 illustrates experimental negative mode MALDI-TOF mass spectrograph data for FDSP-EGCG. The mass peak at 723 m/z corresponds to the molecular ion fragment of fullerene C60 adduct with three residual hydrogen atoms, a unique feature of the main molecular spallation ion of this product. The complicated sharp rider peaks are attributed to the mass fragments of phosphonates as these disassemble from the base fullerene group. The peak at 1419 m/z is attributed to the presence of the non-covalent pi-pi intercalation of EGCG, some of which may be shared between dimeric fullerene functional groups, whereas the peaks centered at about 2042 m/z are attributed to the additional presence of covalently bonded disodium phosphonate groups which are also pendant from the fullerene functional group. The characteristic mass spallation patterns for the illustrated MALDI-TOF data are consistent with and representative of the formation of FDSP-EGCG for this component of the composition according to the teachings of the present invention.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A nanoparticle composition comprising:
buckminsterfullerene (C60) bonded to a molecule that either includes at least one galloyl group or is epicatechin.

2. The nanoparticle composition of claim 1 wherein a bond between the C60 and the molecule is a pi bond.

3. The nanoparticle composition of claim 1 wherein the molecule comprises a quinic acid ester having either di-hydroxyphenyl groups or tri-hydroxy phenyl groups.

4. The nanoparticle composition of claim 1 wherein the molecule comprises epicatechin gallate, epigallocatechin, or epigallocatechin gallate.

5. The nanoparticle composition of claim 1 wherein the molecule comprises penta-m-digalloyl-glucose.

6. The nanoparticle composition of claim 1 wherein the C60 is further bonded to a disodium phosphonate functional group.

7. The nanoparticle composition of claim 6 wherein the C60 is bonded to the disodium phosphonate functional group and further bonded to four additional phosphonate functional groups.

8. The nanoparticle composition of claim 1 further comprising a zeolite, wherein the C60 bonded to the molecule is disposed within the zeolite.

9. The nanoparticle composition of claim 1 further comprising diatomaceous earth, wherein the C60 bonded to the molecule is disposed within porous diatom particles of the diatomaceous earth.

10. The nanoparticle composition of claim 1 further comprising a solvent, wherein the C60 bonded to the molecule is disposed in the solvent.

11. The nanoparticle composition of claim 10 wherein the solvent comprises a mixture of 70% glycerol and 30% polypropylene glycol by volume.

12. A method of curing, treating, or prophylactically avoiding chronic respiratory illnesses including valley fever or COPD, or curing, treating, or prophylactically avoiding an antibiotic resistant strain of bacteria in a subject, the method comprising the step of: administering to the subject an effective amount of a composition including a buckminsterfullerene (C60) bonded to a molecule that either includes at least one galloyl group or is epicatechin.

13. The method of claim 12 wherein the composition includes a pharmaceutically acceptable carrier and the C60 bonded to the molecule is disposed in the pharmaceutically acceptable carrier.

14. The method of claim 13 wherein the pharmaceutically acceptable carrier comprises a zeolite or diatomaceous earth.

15. The method of claim 13 wherein the composition disposed in the pharmaceutically acceptable carrier comprises a tablet, capsule, pill, powder, granule, or a liquid.

16. The method of claim 12 wherein administering the composition comprises administration by an intravenous, intramuscular, subcutaneous, intrathecal, intraperitoneal, topical, nasal, or oral route.

17. The method of claim 12 wherein administering the composition comprises administering an oral dosage including up to about 500 mg of the C60 bonded to the molecule.

18. The method of claim 12 wherein administering the composition comprises administering an intramuscular, intravenous, or a subcutaneous dose of the C60 bonded to the molecule in an amount of from about 0.1 mg/Kg to about 5 mg/Kg.

19. The method of claim 12 wherein administering the composition comprises administering a nano aerosol, a vapor, a powder, a dust, or an aerosolized inhalant.

20. The method of claim 12 wherein the molecule comprises epicatechin gallate, epigallocatechin, or epigallocatechin gallate.

21. The method of claim 12 wherein the C60 is further bonded to a disodium phosphonate functional group.

22. A method of making a nanoparticle, the method comprising:
bonding a buckminsterfullerene (C60) to a molecule that either includes at least one galloyl group or is epicatechin.

23. The method of claim 22 wherein the molecule comprises epicatechin gallate, epigallocatechin, or epigallocatechin gallate.

24. The method of claim 22 wherein the C60 is further bonded to a disodium phosphonate functional group.

25. The method of claim 22 wherein bonding the disodium phosphonate functional group to the C60 is performed by reaction shear mixing.

26. The method of claim 22 wherein bonding the molecule to the C60 is performed by reaction shear mixing.

27. The method of claim 22 wherein bonding the molecule to the C60 is performed at no more than about 55° C.
